(19) United States
(12) Patent Application Publication
(10) Pub. No.: US 2014/0213520 A1
(43) Pub. Date: Jul. 31, 2014

(54) METHODS OF TREATING CARDIOVASCULAR INDICATIONS

(71) Applicant: Cardiorentis Ltd., Zug (CH)

(72) Inventor: JOHANNES HOLZMEISTER, ZURICH (CH)

(73) Assignee: Cardiorentis Ltd., Zug (CH)

(21) Appl. No.: 14/163,273

(22) Filed: Jan. 24, 2014

Related U.S. Application Data
(60) Provisional application No. 61/756,692, filed on Jan. 25, 2013.

(51) Int. Cl. A61K 38/22

(52) U.S. Cl. CPC A61K 38/2242; A61K 38/2221
USPC 514/12.4; 514/12.7

(57) ABSTRACT

Disclosed in certain embodiments is a method of treating a cardiovascular indication comprising administering a natriuretic peptide to a patient in need thereof within 24 hours of clinical assessment of the patient.

### BACKGROUND OF THE INVENTION

This application claims priority to U.S. Provisional Application Ser. No. 61/756,692 filed Jan. 25, 2013, the disclosure of which is hereby incorporated by reference.

A family of related peptides has been discovered that works in concert to achieve salt and water homeostasis in the body. These peptides, termed natriuretic peptides for their role in moderating natriuresis and diuresis, have varying amino acid sequences and originate from different tissues within the body. This family of natriuretic peptides consists of atrial natriuretic peptide (“ANP”), brain natriuretic peptide (“BNP”), C-type natriuretic peptide (“CNP”), Dendroaspis natriuretic peptide (“DNP”), and urodilatin (“URO”, or ularitide). Their tissue-specific distribution of these peptides is as follows: heart (ANP, BNP, and DNP); brain (ANP, BNP, and CNP); endothelial cells (CNP); plasma (DNP); and kidney (URO). These peptides are constituents of a hormonal system that plays a critical role in maintaining an intricate balance of blood volume/pressure in the human body. For instance, urodilatin, a close analog of ANP secreted by kidney tubular cells, promotes excretion of sodium and water by acting directly on kidney cells in the collecting duct to inhibit sodium and water reabsorption.

Like other natriuretic peptides, such as ANP and BNP, urodilatin has been studied for use in treating various conditions, including renal failure and cardiovascular conditions such as congestive heart failure (see, e.g., U.S. Pat. Nos. 5,571,789 and 6,831,064; Kentsch et al., Eur. J. Clin. Invest. 1992, 22(10):662-669; Kentsch et al., Eur. J. Clin. Invest. 1995, 25(4):281-283; Elsner et al., Am. Heart J. 1995, 129(4):766-773; and Forssmann et al., Clinical Pharmacology and Therapeutics 1998, 64(3):322-330).

Cardiovascular diseases are a leading cause of death, regardless of gender or ethnicity. Among these diseases, congestive heart failure (“CHF”) is highly prevalent. According to the American Heart Association, the number of hospital discharges and the number of deaths due to CHF both rose roughly 2.5-fold from 1979 to 1999. Currently, about 5 million Americans have been diagnosed with CHF, and about 550,000 new cases occur annually (American Heart Association 2001). This life-threatening condition is accompanied by great financial impact.

There continues to be a need for new and more effective methods for treating cardiovascular conditions, especially in the area of acute onset of symptoms in an emergency situation.

All documents referenced herein are hereby incorporated by reference in their entireties for all purposes.

### OBJECTS AND SUMMARY OF THE INVENTION

It is an object of the present invention to provide methods for the treatment of cardiovascular events, e.g., acute onset cardiovascular events.

The objects are met by the present invention which in certain embodiments is directed to a method of treating a cardiovascular indication comprising administering a natriuretic peptide, a diuretic peptide and/or a vasodilatory peptide to a patient in need thereof within 24 hours of clinical assessment of the patient.

In one embodiment, the methods of the present invention may result in the prevention or minimization of myocardial cell death. The prevention or minimization of this cell death may be in the presence of one or more factors selected from C-reactive protein, TNF-alpha, IL-1β, endothelin-1 or galectin-3.

In another embodiment, the methods of the present invention may result in the prevention or minimization of nitrosylation of myocardial cells. The prevention or minimization of nitrosylation of myocardial cells may be in the presence of one or more factors selected from C-reactive protein, TNF-alpha, IL-1β, endothelin-1 or galectin-3

In certain embodiments, the present invention is directed to a use of a natriuretic peptide, a diuretic peptide and/or a vasodilatory peptide for the treatment of a cardiovascular indication on a patient in need thereof within 24 hours of clinical assessment of the patient.

In certain embodiments, the present invention is directed to a use of a natriuretic peptide, a diuretic peptide and/or a vasodilatory peptide in the preparation of a medicament for the treatment of a cardiovascular indication on a patient in need thereof within 24 hours of clinical assessment of the patient.

In certain embodiments, the present invention is directed to a use of a natriuretic peptide, a diuretic peptide and/or a vasodilatory peptide to prevent or minimize myocardial cell death. In one embodiment, the use to prevent or minimize myocardial cell death is in the presence of one or more factors selected from C-reactive protein, TNF-alpha, IL-1β, endothelin-1 or galectin-3. In another embodiment, the use to prevent or minimize myocardial cell death is within about 2 hours, within about 4 hours, within about 6 hours, within about 10 hours or within about 24 hours of exposure to the one or more factors.

In certain embodiments, the present invention is directed to a use of a natriuretic peptide, a diuretic peptide and/or a vasodilatory peptide to prevent or minimize nitrosylation of myocardial cells. In one embodiment, the use to prevent or minimize nitrosylation of myocardial cells is in the presence of one or more factors selected from C-reactive protein, TNF-alpha, IL-1β, endothelin-1, or galectin-3. In another embodiment, the use to prevent or minimize nitrosylation of myocardial cells is within about 2 hours, within about 4 hours, within about 6 hours, within about 10 hours or within about 24 hours of exposure to the one or more factors.

In certain embodiments, the present invention is directed to a kit comprising a natriuretic peptide, a diuretic peptide and/or a vasodilatory peptide and instructions for use in the treatment of a cardiovascular indication on a patient in need thereof within 24 hours of clinical assessment of the patient.

In certain embodiments, the natriuretic peptide utilized in the present invention is ularitide or neseritide.

As used herein, the term “cardiovascular indication” encompasses all types of cardiovascular conditions that, regardless of their cause, are generally recognized by a physician as heart failure, which include but are not limited to, acute heart failure, chronic heart failure, congestive heart failure (CHF), and particularly acute decompensated heart failure (which is a separate and distinct disease state than CHF). In this application, the terms acute decompensated heart failure (“ADHF”) and decompensated heart failure (“DHF”) are used interchangeably. These conditions typically involve weakened heart function combined with a build-up of body fluid and may be the result of either a sudden event, such as myocardial infarction or the rupture of a heart valve, or a chronic and slowly progressing process, such as the gradual weakening of heart muscles due to cardiomyopathy from infections or alcohol/drug abuse, and other pre-existing medical conditions such as hypertension, coronary artery disease, valve disease, thyroid disease, kidney disease, diabetes, or congenital heart defects. Also encompassed by the term “heart failure” are any heart conditions relating to fluid build-up in the heart, such as myocardial edema.

The term “administrate” or “administration,” as used herein, encompasses various methods of delivering a composition containing a natriuretic peptide to a patient. Modes of administration may include, but are not limited to, methods that involve delivering the composition intravenously, intraperitoneally, intranasally, transdermally, topically, subcutaneously, parentally, intramuscularly, orally, or systemically, and via injection, ingestion, inhalation, implantation, or adsorption by any other means. The preferred means of administering a composition comprising a natriuretic peptide (e.g., ularitide) is intravenous injection, where the composition is formulated as a sterile solution. Another route of administration is oral ingestion, where the natriuretic peptide can be formulated as a pharmaceutical composition in the form of a syrup, an elixir, a suspension, a powder, a granule, a tablet, a capsule, a lozenge, a troche, an aqueous solution, a cream, an ointment, a lotion, a gel or an emulsion. In some embodiments, the pharmaceutical composition for oral ingestion is formulated for sustained release over a period of at least 24 hours. Furthermore, administration of a natriuretic peptide can be achieved by subcutaneous injection of a natriuretic peptide-containing composition, which is prepared as a sustained release system comprising microspheres or biodegradable polymers, such that the natriuretic peptide can be released into a patient's body at a controlled rate over a period of time, e.g., at least 24 hours or 48 hours.

An “effective amount” refers to the amount of an active ingredient, e.g., urodilatin, in a pharmaceutical composition that is sufficient to produce a beneficial or desired effect at a level that is readily detectable by a method commonly used for detection of such an effect. In some embodiments, such an effect results in a change of at least 10% from the value of a basal level where the active ingredient is not administered. In other embodiments, the change is at least 20%, 50%, 80%, or an even higher percentage from the basal level. As will be described below, the effective amount of an active ingredient may vary from subject to subject, depending on age, general condition of the subject, the severity of the condition being treated, and the particular biologically active agent administered and the like. An appropriate “effective” amount in any individual case may be determined by one of ordinary skill in the art by reference to the pertinent texts and literature and/or by using routine experimentation.

The term “natriuretic peptide” refers to a peptide that has the biological activity of promoting natriuresis, diuresis and vasodilation. Assays for testing such activity are known in the art, e.g., as described in U.S. Pat. Nos. 4,751,284 and 5,449,751. Examples of natriuretic peptides include, but are not limited to, atrial natriuretic peptide (ANP(99-126)), brain natriuretic peptide (BNP), C-type natriuretic peptide (CNP), Dendroaspis natriuretic peptide (DNP), urodilatin (URO, or ularitide), and any fragments of the prohormone ANP(1-126) or BNP precursor polypeptide that retains the vasodilating, natriuretic or diuretic activity. For further description of exemplary natriuretic peptides and their use or preparation, see, e.g., U.S. Pat. Nos. 4,751,284, 4,782,044, 4,895,932, 5,449,751, 5,461,142, 5,571,789, and 5,767,239. See also, Ha et al., Regul. Pept. 133(1-3):13-19, 2006. The invention can also be practiced with peptides and proteins which may cause a diuresis or vasodilation effect such as relaxin.

As used in this application, the term “urodilatin” refers to a 32-amino acid peptide hormone that is described by U.S. Pat. No. 5,449,751 and has the amino acid sequence set forth in GenBank Accession No. 1506430A. Urodilatin, the 95-126 fragment of atrial natriuretic peptide (ANP), is also referred to as ANP(95-126). The term “atrial natriuretic peptide” or “ANP(99-126)” refers to a 28-amino acid peptide hormone, which is transcribed from the same gene and derived from the same polypeptide precursor, ANP(1-126), as urodilatin but without the first four amino acids at the N-terminus. For a detailed description of the prohormone, see, e.g., Oikawa et al. (Nature 1984; 309:724-726), Nakayama et al. (Nature 1984; 310:699-701), Greenberg et al. (Nature 1984; 312:656-658), Seidman et al. (Hypertension 1985; 7:31-34) and GenBank Accession Nos. 1007205A, 1009248A, 1101403A and AAA35529.

Conventionally, the term urodilatin (URO) is more often used to refer to the naturally occurring peptide, whereas the term ularitide is often used to refer to the recombinantly produced or chemically synthesized peptide. In this application, the term “urodilatin” and “ularitide” are used interchangeably to broadly encompass both a naturally occurring peptide and a recombinant or synthetic peptide. The terms also encompass any peptide of the above-cited amino acid sequence containing chemical modification (e.g., deamination, phosphorylation, PEGylation, etc.) at one or more residues or substitution by the corresponding D-isomer(s), so long as the peptide retains the biological activity as a natriuretic peptide. Furthermore, a chemically modified urodilatin or ularitide may contain one or two amino acid substitutions for the purpose of facilitating the desired chemical modification (e.g., to provide a reactive group for conjugation). “Urodilatin” or “ularitide” of this application, regardless of whether it contains chemical modifications, retains a substantial portion, i.e., at least 50%, preferably at least 80%, and more preferably at least 90%, of the biological activity of the naturally-occurring wild-type urodilatin or ANP(95-126).

The term “cardiac medicine” refers to a therapeutic agent that is useful for treating a cardiac condition. A “cardiac medicine” includes but is not limited to natriuretic peptides, ACE inhibitors (“ACEIs”), beta-adrenergic blocking agents (“beta-blockers”), vasodilators, diuretics, digitalis preparations (e.g., digoxin), dopamine, dobutamine, levosimendan, nesiritide, blood thinners, angiotensin II receptor blockers, calcium channel blockers, nitrates and potassium.

The term “pharmaceutically acceptable excipient or carrier” refers to any inert ingredient in a composition that may act, for example, to stabilize the active ingredient. A pharmaceutically acceptable excipient can include, but is not limited to, carbohydrates (such as glucose, sucrose, or dextrans), antioxidants (such as ascorbic acid or glutathione), chelating agents, low-molecular weight proteins, high-molecular weight polymers, gel-forming agents or other stabilizers and additives. Other examples of a pharmaceutically acceptable carrier include wetting agents, emulsifying agents, dispersing agents or preservatives, which are particularly useful for preventing the growth or action of microorganisms. Various preservatives are well known and include, for example, phenol and ascorbic acid. Examples of carriers, stabilizers or adjuvants can be found in Remington's Pharmaceutical Sciences, Mack Publishing Company, Philadelphia, Pa., 17th ed. (1985).

As used herein, a “patient” refers to a human or a non-human mammal.

### BRIEF DESCRIPTION OF THE FIGURES

FIG. 1 depicts the percent of myocardial cell death according to the assays of Example 2.

FIG. 2 depicts the nitrosylation of myocardial cells according to the assays of Example 2.

FIG. 3 depicts the results of a lactate dehydrogenase assay of Example 2.

### DETAILED DESCRIPTION

The present invention is directed to a method of treating a cardiovascular indication comprising administering a natriuretic peptide, a diuretic peptide or a vasodilatory peptide to a patient in need thereof within 24 hours of clinical assessment of the patient. By virtue of the present invention, the early treatment with these agents within the time frame may result in improved outcomes (e.g., by preserving myocardial cells) compared to late treatment, outside of the time frame.

An early intervention with a natriuretic peptide may cause a reduction in cardiac wall stress and myocardial injury at a critical time. Lowering intracardiac filling pressure early, e.g., within 24 hours, may result in better protection than late intervention. The resulting salvage of myocardium by the methods of the present invention become manifest as a favorable effect on clinical outcome.

The early intervention of the present invention may be within 24 hours, within 22 hours, within 20 hours, within 18 hours, within 16 hours, within 14 hours, within 12 hours, within 10 hours, within 8 hours, within 6 hours, within 5 hours, within 4 hours, within 3 hours, within 2 hours, within 1 hour or within 30 minutes of clinical assessment of the patient.

Upon initiation of therapy, the administration may be an immediate administration (e.g. by a parenteral bolus) or continuous over a time period of at least about 2 hours, at least about 3 hours, at least about 4 hours, at least about 5 hours, at least about 6 hours, at least about 8 hours, at least about 10 hours, at least about 12 hours, at least about 14 hours, at least about 16 hours, at least about 18 hours, at least about 20 hours, at least about 22 hours, at least about 24 hours, at least about 30 hours, at least about 32 hours, at least about 36 hours, at least about 40 hours, at least about 44 hours or at least about 48 hours. In certain embodiments, the duration is from about 2 hours to about 120 hours, from about 2 hours to about 48 hours, from about 2 hours to about 24 hours, from about 12 hours to about 120 hours, and in other embodiments, from about 24 hours to about 96 hours, or from about 24 hours to about 72 hours, or from about 36 hours to about 60 hours, or from about 40 hours to about 56 hours, or from about 44 hours to about 52 hours, or from about 46 hours to about 50 hours or about 48 hours. A preferred means for administering the peptide (e.g., the natriuretic peptide) is by parenteral (e.g., intravenous) administration.

When the peptide (e.g., the natriuretic peptide) is administered parenterally, the administration can be, e.g., by injection or infusion. When the parenteral administration is by injection, the route can be intravenous (into a vein), subcutaneous (under the skin), intramuscular (into muscle), intraperitoneal, intravitreal (intraocular), intracerebral or intraspinal. When the parenteral administration is by infusion, it is typically by an intravenous route. The parenteral administration can be by a sterile dosage form that is a solution, suspension or emulsion. For the present invention, the peptide (e.g., natriuretic peptide) can be formulated for administration by a variety of techniques, including, for example, subcutaneous, intravenous, oral, rectal, transmucosal, transdermal, intestinal, parenteral, intramuscular, intramedullary, intrathecal, direct intraventricular, intraperitoneal, intranasal, and intraocular administration, among others.

When the peptide (e.g., the natriuretic peptide) is administered enterally, the administration can be, e.g., oral, sublingual (dissolving the drug under the tongue) or rectal.

In one embodiment of the invention, the natriuretic peptide used in the method is ularitide or urodilatin. Alternatively, the natriuretic peptide may be atrial natriuretic peptide (ANP), brain natriuretic peptide (BNP), C-type natriuretic peptide (CNP), Dendroaspis natriuretic peptide (DNP) or neseritide. The invention can also be practiced with other vasodilatory peptides such as relaxin.

In another embodiment, one or more different cardiac medicines are administered to the patient. These one or more different cardiac medicines may be administered in combination with the natriuretic peptide (e.g., urodilatin), for example, by the same route (e.g., intravenously), with the option of being in one single pharmaceutical composition or two or more separate compositions; or these one or more different cardiac medicines may be administered separately by a different means (e.g., by oral ingestion).

The composition used in the method of this invention optionally further comprises a pharmaceutically acceptable excipient or carrier. For example, mannitol may be used in such a pharmaceutical composition. In an exemplary embodiment, the concentration of mannitol is 2 times, 3 times, 4 times, 5 times, 6 times, 7 times, 8 times, 9 times or 10 times the concentration of the peptide, such as urodilatin. In another exemplary embodiment, the composition is an aqueous solution of 0.9% NaCl in which the peptide, such as urodilatin, is dissolved. In one particular embodiment of the method, the composition is an aqueous solution of 0.9% NaCl in which urodilatin and mannitol are dissolved, the rate of urodilatin infusion is 15 ng/kg/min, and the time period for continuous infusion is 48 hours.

In another aspect, the present invention provides the use of a peptide, such as urodilatin, for the manufacture of a medicament for the treatment of heart failure, which includes acute decompensated heart failure and chronic congestive heart failure, in accordance with the present invention. The medicament may contain, in addition to an effective amount of the active ingredient (e.g., a natriuretic peptide, such as urodilatin), a pharmaceutically acceptable excipient or carrier. In one embodiment, the medicament is formulated for continuous intravenous administration over a time period of at least 12 hours. In other embodiments, the medicament is formulated for continuous intravenous administration over a time period of 24 hours to 120 hours. In some cases, the medicament is formulated for a sustained release of the peptide over a period of at least 12 hours, e.g., about 24 to 72 hours or 48 to 72 hours. For example, the administration of the peptide-containing medicament may last about 24 hours, about 36 hours, about 48 hours, about 60 hours, about 72 hours, about 96 hours, about 120 hours or any desirable time duration within this range.

In some embodiments, the medicament is administered in a manner such that the patient is receiving the active ingredient (e.g., urodilatin) at a rate of at least about 1 ng/kg/minute, of at least about 2 ng/kg/minute, of at least about 5 ng/kg/minute, of at least about 7.5 ng/kg/minute, of at least about 10 ng/kg/minute of at least about 15 ng/kg/minute, of at least about 20 ng/kg/minute, of at least about 30 ng/kg/minute, of at least about 45 ng/kg/minute, of at least about 60 ng/kg/minute, of at least about 75 ng/kg/minute of at least about 100 ng/kg/minute, or of at least about 200 ng/kg/minute. In other embodiments, the administration rate is about 7.5 ng/kg/ minute, about 15 ng/kg/minute or about 30 ng/kg/minute. In one preferred example, ularitide is administered at the rate of about 15 ng/kg/minute.

The methods of the present invention can be utilized to treat, e.g., heart failure, acute heart failure, chronic heart failure, congestive heart failure, acute decompensated heart failure, abnormal fluid accumulation in the heart, myocardial edema, dyspnea or any combination thereof.

The administration of a peptide (e.g., natriuretic peptide) according to the present invention is preferably achieved by intravenous injection, subcutaneous injection or oral ingestion. For intravenous administration, the composition comprising, e.g., a natriuretic peptide, may be formulated with an aqueous diluent, suitably mixed with other optional additives such as a surfactant and/or a preservative for proper fluidity, stability and sterility of the composition, necessary for easy storage and injection. The injectable solution containing a peptide may be prepared using a solvent or dispersion medium including water, ethanol, polyol (e.g., glycerol, propylene glycol, liquid polyethylene glycol and the like), suitable mixtures thereof, and/or vegetable oils. Proper fluidity may be maintained, for example, by the use of a coating material, such as lecithin, by the maintenance of the required particle size in the case of dispersion and by the use of surfactants. The prevention of the proliferation of microorganisms can be facilitated by various antibacterial and antifungal agents, for example, parabens, chlorobutanol, phenol, sorbic acid, thimerosal and the like. In many cases, it is preferable to include isotonic agents, for example, sugars or sodium chloride. Prolonged absorption of the injectable compositions can be brought about by the use in the compositions of agents delaying absorption, for example, aluminum monostearate and gelatin. The injectable solution should be suitably buffered if necessary and the liquid diluent first rendered isotonic with sufficient saline or glucose. Lastly, the injectable solution, once prepared by incorporating the active ingredients in the required amount in the appropriate solvent with optional excipients, is sterilized using a method that does not inactivate the active ingredient(s) of the composition, e.g., by filtered sterilization.

As disclosed herein, the peptide, e.g., a natriuretic peptide, can be formulated with mannitol. Non-limiting examples of other sugars that may be used in embodiments of the present invention include abequose, allose, allulose, altrose, apiose, arabinose, beet oligosaccharides, bifurcose, deoxyribose, dextrose(D-glucose), erlose, erythrose, erythrulose, fructose (levulose), fucose, fuculose, galactose, gentiobiose, gentiotriose, gentiotetraose, gulose, hamamelose, inulobiose, inulotriose, inulotetraose, isomaltose, isomaltotriose, isomaltotetraose, isomaltopentaose, isomaltulose (palatinose), kestose, kojibiose, lactose, lactulose, laminaribiose, lyxose, mannose, maltose, maltotriose, maltotetraose, maltulose, meletzitose, melibiose, methose, nigerose, nystose, panose, paratose, primeverose, psicose, raffinose, rhamnose, ribose, ribulose, rutinose, sorbinose, sorbose, soybean oligosaccharides, stachyose, sucrose, tagatose, talose, theanderose, threose, trehalose, turanose, xylobiose, xylotriose, xylose or xylulose. The carbohydrates used in embodiments of the present invention may be of their respective D- or L-configurations.

In certain embodiments, non-limiting examples of sugar alcohols that may be used include allitol, arabitol, erythritol, galactitol, glycerol, glycol, iditol, inositol, isomalt, lactitol, maltotetraol, maltotriol, ribitol, sorbitol, talitol, threitol and xylitol. The sugar alcohols used in embodiments according to the present invention may be of their respective the D- or L-configurations. These sugar alcohols have the benefits of having low glycemic indices. Mannitol, for example, has been used to treat increased intracranial pressure.

For oral administration, the composition comprising a natriuretic peptide may be formulated with an inert diluent or other pharmaceutically acceptable excipient, or it may be enclosed in a hard- or soft-shell gelatin capsule, or it may be compressed into tablets. The active ingredients (e.g., ularitide) may be incorporated with excipients and used in the form of ingestible tablets, buccal tables, troches, capsules, caplets, elixirs, suspensions, syrups, wafers and the like. The orally ingestible formulation preferably contains high-molecular weight polymers or gel-forming agents that allow sustained release of the natriuretic peptide over an extended period of time, for example, at least 8 hours, at least 12 hours or at least 24 hours. This sustained release system achieves the slow release of the active ingredient over a period of time, either as a controlled release system, which is effective in maintaining substantially constant level of the natriuretic peptide (e.g., urodilatin) in the blood, or as a prolonged release system, which, although unsuccessful at achieving substantially constant blood level of a natriuretic peptide, but nevertheless extends the duration of action of the natriuretic peptide over that time period.

### EXAMPLES

### Example 1

A Phase III, Multicenter, Randomized, Double-Blind, Placebo-Controlled Trial to Evaluate the Efficacy and Safety of Ularitide (Urodilatin) Intravenous Infusion in Patients Suffering from Acute Decompensated Heart Failure is initiated as follows

### Clinical Study Design:

Prospective, randomized, placebo-controlled, double-blind, multinational, multi-center study.

### Number of Study Sites:

Approximately 120 centers in North America, Europe and Latin America

### Type and Number of Patients:

Approximately 2,2500 patients with acute decompensated heart failure (ADHF) Objective:

To evaluate the effect of a continuous intravenous (“IV”) ularitide infusion on the clinical status of patients with ADHF.

### Primary Efficacy Endpoints:

There are two co-primary endpoints. Co-Primary Efficacy Endpoint 1 evaluates changes in a hierarchical clinical composite comprised of elements associated with: patient global assessment using a 7-point scale of symptomatic change; lack of improvement, or worsening; persistent or worsening heart failure (HF) as documented by signs and symptoms and requiring an intervention (initiation or intensification of IV therapy, circulatory or ventilatory mechanical support, surgical intervention, ultrafiltration, hemofiltration or dialysis); and all-cause mortality. Assessment of the clinical composite will be performed at 6 hour (“h”), 24 h and 48 h after start of IV ularitide infusion.

Patients will be classified as “improved” if the patients are moderately or markedly improved at all three time points (at 6 h, 24 h and 48 h) and do not fulfill criteria for “worse” during the first 48 hours following the start of the study drug infusion. Patients will be classified as “worse” if (during the 48 h) they die; experience worsening HF requiring a pre-specified intervention at any time during the first 48 h; or experienced moderate or marked worsening of their global assessment at any of the three time points (at 6 h, 24 h or 48 h).

Co-Primary Efficacy Endpoint 2 evaluates cardiovascular mortality during follow-up after randomization for the entire duration of the trial.

### Primary Safety Endpoint:

All-cause mortality and cardiovascular re-hospitalization at 30 days after start of study drug infusion.

### Secondary Endpoints:

Changes of N-terminal pro brain natriuretic peptide (NT-pro BNP) at 48 h of treatment compared to baseline.
All-cause mortality and cardiovascular re-hospitalization at Day 90 after start of study drug infusion.

### Exploratory Endpoints:

Components of primary efficacy endpoint:
   a. Proportions Improved/Not Improved and Worse/Not Worse,
   b. Proportions of patients alive,
   c. Proportions of patients requiring an intervention for persistent or worsening heart failure,
   d. Proportions of patients who are “moderately or markedly improved”.
Combined risk of all-cause mortality or cardiovascular re-hospitalization at Day 60 and Day 180 after start of study drug infusion.
Changes in blood pressure (“BP”) and heart rate during the first 72 h from the start of the study drug infusion or hospital discharge, whatever comes first.
Length of stay of index hospitalization in hours after start of study drug infusion.

Change in glomerular filtration rate (“GFR”) as assessed by Modification of Diet in Renal Disease (“MDRD”) at 48 h after start of study drug infusion as compared to baseline.

### Inclusion Criteria:

1) Males and females aged 18 to 85 years.
2) Unplanned hospitalization or emergency department visit for ADHF. Acute HF is defined as including all of the following:
   a) Dyspnea at rest in a recumbent sitting position (30 to 45 degrees), which has worsened within the past week.
   b) Radiological evidence of HF on a chest X-ray.
   c) BNP >500 pg/mL or NT-pro BNP >2000 pg/mL.
3) Ability to start infusion of the study drug within 12 h after initial clinical assessment performed by a physician at the emergency room/hospital with symptoms of ADHF.
4) Ability to reliably carry out self-assessment of symptoms.
5) Systolic blood pressure (“SBP”) ≧110 mmHg.
6) Persisting dyspnea at rest despite standard background therapy for ADHF (as determined by the Investigator) which must include IV furosemide (or equivalent diuretic) at ≧40 mg (or its equivalent) at any time after start of emergency services (ambulance, emergency department, or hospital). At the time of randomization, the patient must still be symptomatic. In addition, the patient should not have received an IV bolus of a diuretic for at least 2 h prior to randomization, and the infusion rates of ongoing IV infusions must not have been increased or decreased for at least 2 h prior to randomization.
7) Ability to understand the purpose and risks of the study and provide signed and dated informed consent and authorization to use protected health information (in accordance with national and local privacy regulations).

### Exclusion Criteria:

1) Known active myocarditis, obstructive hypertrophic cardiomyopathy, congenital heart disease, restrictive cardiomyopathy, constrictive pericarditis or uncorrected clinically significant primary valvular disease.
2) Treatment with dobutamine at a dose >5 μg/kg/min or use of drugs for support of BP at the time of randomization.
3) Treatment with levosimendan, milrinone or any other phosphodiesterase inhibitor within 7 days before randomization.
4) Treatment with nesiritide within 30 days before randomization.
5) Creatinine clearance <30 mL/min/1.73 m² (as measured by the MDRD formula) at the time of screening.
6) Planned coronary revascularization procedure (percutaneous coronary intervention or coronary artery bypass grafting) within 5 days of randomization.
7) Clinical diagnosis of acute coronary syndrome meeting any two of the following three criteria:
   a) Prolonged chest pain at rest or an accelerated pattern of angina;
   b) Electrocardiogram (“ECG”) changes indicative of ischemia or myocardial injury;
   c) Serum troponin >3 times upper limit of normal.
8) Clinically suspected acute mechanical cause of ADHF (e.g., papillary muscular rupture). The diagnosis need not be confirmed by imaging or cardiac catheterization.
9) Anemia (hemoglobin <9 g/dL or a hematocrit <25%).
10) Known vasculitis, active infective endocarditis or suspected infections including pneumonia, acute hepatitis, systemic inflammatory response syndrome or sepsis.
11) Body temperature ≧38° C. just prior to randomization.
12) Acute or chronic respiratory disorder (e.g. severe chronic obstructive pulmonary disease) or primary pulmonary hypertension sufficient to cause dyspnea at rest, which may interfere with the ability to interpret dyspnea assessments or hemodynamic measurements.
13) Terminal illness other than congestive heart failure with expected survival <180 days.
14) Any previous exposure to ularitide.
15) Known allergy to natriuretic peptides.
16) Participation in an investigational clinical drug trial within 30 days prior to randomization.
17) Current drug abuse or chronic alcoholism sufficient to impair participation and compliance to the study protocol.
18) Women who are breast-feeding.
19) Women of child-bearing potential without documentation of a negative urine pregnancy assay within 12 h prior to randomization.
20) Any condition that, in the Investigator's opinion, makes the patient unsuitable for study participation.
21) Legal incapacity or limited legal capacity.
22) Implanted Left Ventricular Assist Device (LVAD)

### Investigational Medicinal Product:

Ularitide for injection. Ularitide, a natriuretic peptide, is lyophilized with mannitol (2.5 mg ularitide with 20 mg mannitol) in labeled 10 mL vials

### Reference Therapy:

Matching placebo, i.e., 20 mg mannitol in vials that are identical to the ularitide vials to maintain blinding

Dose, Mode and Duration of treatment:

Continuous IV infusion of randomly assigned placebo or ularitide 15 ng/kg/minute will be initiated after randomization and continued for 48 h. The body weight (“BW”)-adjusted dose will be the same for all patients with a BW >115 kg corresponding to a maximal total daily dose of 2.484 mg/day.

A dose of 15 ng/kg/min of ularitide has been chosen because in previous studies in HF patients, the hemodynamic and clinical benefits of a 24-h infusion of 15 ng/kg/min infusion were similar to those of 30 ng/kg/min, but superior to those observed with 7.5 ng/kg/min infusion of ularitide. Infusion of 15 ng/kg/min was better tolerated than the infusion of 30 ng/kg/min

### Study Design:

Patients with ADHF who meet all inclusion and exclusion criteria will be randomized on a 1:1 basis to continuous IV infusion of either ularitide 15 ng/kg/min or matching placebo for 48 h. In addition, patients may receive all appropriate therapy that may include vasodilatory, inotropic and diuretic support as clinically indicated, but investigators should not make the diagnosis of or intervene for persistent heart failure for at least 6 hours following randomization, in order to allow the effects of the study medication to become apparent. In addition, use of nesiritide, levosimendan, milrinone, or any other phosphodiesterase inhibitor is not allowed during the first 72 h following the start of the infusion.

All timepoints refer to the start of the study drug infusion at the timepoint called “0 hours” (t₀). Co-primary Efficacy Endpoint 1 will be assessed at 6 h, 24 h and 48 h from the start of infusion. Co-primary Efficacy Endpoint 2 will be assessed during follow-up after randomization.

Safety parameters will be assessed during hospitalization and adverse events (“AEs”) and serious adverse events (“SAEs”) will be evaluated until Day 30 after the start of therapy.

All patients will be assessed through a hospital visit at Day 30 and phone call follow-ups at Day 60, Day 90 and Day 180 for the occurrence of cardiovascular re-hospitalization and all-cause mortality.

### Independent Committees:

All outcomes associated with the primary endpoints will be adjudicated by an independent Clinical Events Committee (“CEC”). In addition, all cardiovascular hospitalizations and deaths recorded during the 180-day follow-up period will be adjudicated.

An independent Data and Safety Monitoring Board (“DSMB”) will monitor all efficacy and safety outcomes but will be able to recommend early termination of the trial only for mortality. There is no intent for the trial, however, to be terminated early for a favorable treatment effect on either primary efficacy endpoint

### Statistical Analyses

Co-primary Efficacy Endpoint 1 for this study is a hierarchical composite variable comprised of elements associated with patient global assessment using a 7-point scale of symptomatic improvement, lack of improvement, or worsening: persistent or worsening HF requiring a pre-specified intervention, and all-cause mortality. The composite variable is assessed at 6 h, 24 h and 48 h after the start of IV study drug infusion.

Co-primary efficacy endpoint 2 for this study is freedom from cardiovascular mortality after randomization.

The primary safety variable is the proportion of patients that have died or had a cardiovascular rehospitalization up to Day 30.

If either primary efficacy endpoint and the safety endpoint are met, the following secondary endpoints will be hierarchically tested:
1. Changes in NT-pro BNP from baseline at 48 h of treatment.
2. All-cause mortality and cardiovascular re-hospitalization at Day 90 after the start of IV study drug infusion.
3. Cardiovascular mortality rate at Day 90.

If a patient reports moderate or marked improvement or moderate or marked worsening of their patient global assessment at 6h, 24 h or 48 h, he/she will be asked to identify the symptom or symptoms whose change led him/her to conclude that their patient global assessment had meaningfully changed. The frequency of symptoms which led to improvement or worsening will be compared across the 2 treatment groups.

### Example 2

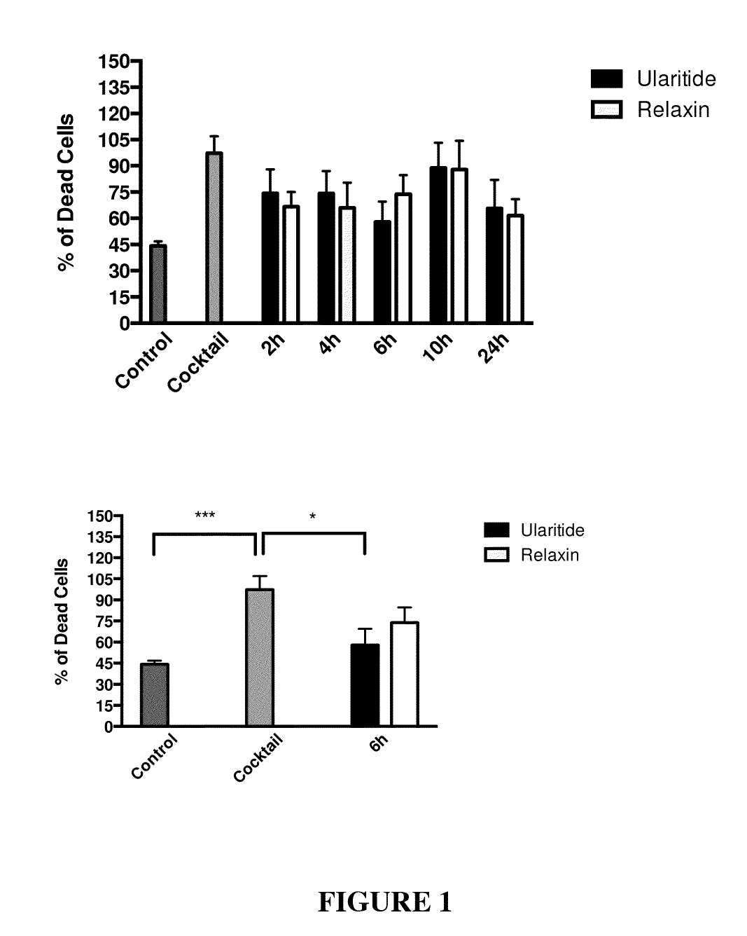

Cardiac microvascular endothelial cells (“HCMEC”) were exposed for 30 hours to a conditioned milieu (“cocktail”) containing a selection of factors (C-reactive protein at 10 μg/ml, TNF-alpha at 10 μg/ml, IL-1β at 10 ng/ml, endothelin-1 at 10 pg/ml and galectin-3 at 20 ng/ml) found in the plasma of ADHF patients. During exposure to the conditioned milieu, HCMEC was treated with ularitide or relaxin at different time points (2 hours, 4 hours, 6 hours, 10 hours and 24 hours). The cells from each exposure were harvested at 30 hours for experimental data. Experimental readouts included apoptosis, total protein nitrosylation and lactate dehydrogenase (“LDH”) assay. The results are set forth in FIGS. 1, 2 and 3.

In the data represented by FIG. 1, apoptosis was assessed by performing an ELISA (enzyme-linked immunosorbent assay) measuring DNA fragmentation. The data demonstrates that ularitide and relaxin both decrease cocktail-induced apoptosis with ularitide showing a greater decrease than relaxin.

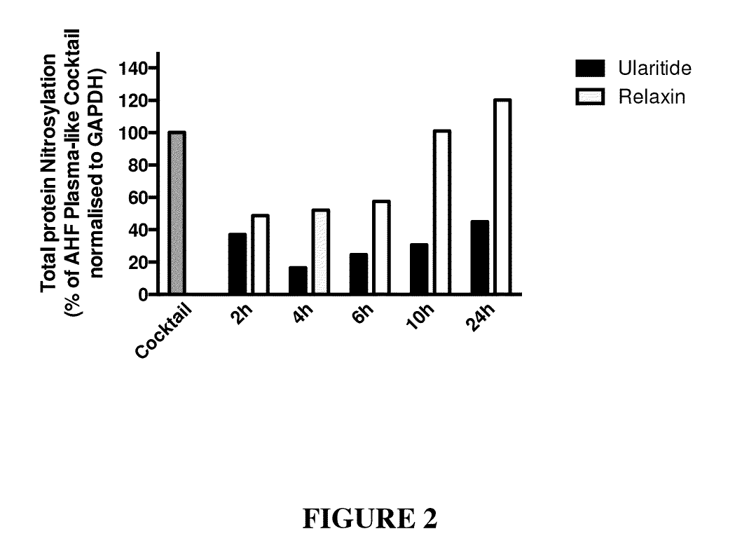

The data represented by FIG. 2 is directed to protein nitrosylation which is a postranlational modification caused by peroxynitrite. Peroxynitrite (ONOO⁻) results from the scavenging action of superoxide anion (O₂⁻) on nitric oxide (NO) and results in a decreased bioavailability of the latter. Protein Nitrosylation has several deleterious effects on key vasoactive factors and their function, e.g. prostacyclin synthase is inactivated by nitrosylation. As demonstrated in FIG. 2, protein nitrosylation was assessed by western blotting and results denote an antioxidant property of ularitide at all time points. Relaxin also shows an antioxidant property that decreases over time.

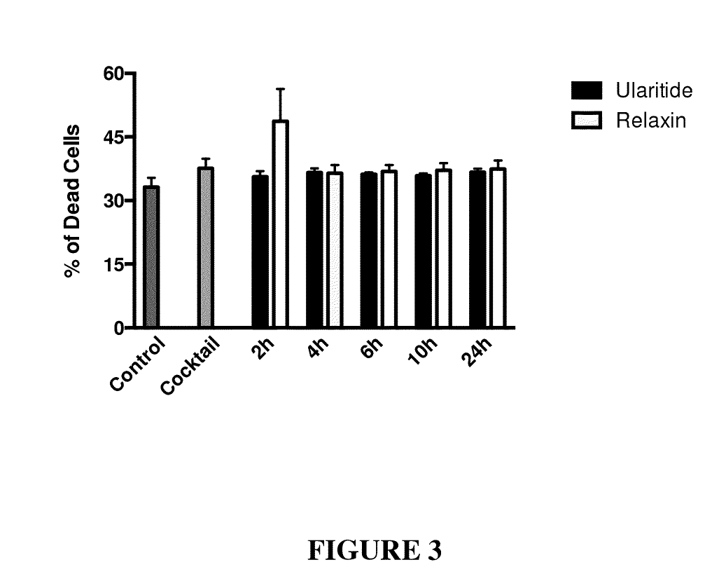

FIG. 3 depicts a LDH assay that was performed by measuring LDH release (by spectrophotometry) in the medium where cells were cultured. LDH is a cytoplasmic enzyme released by cells following membrane damage (i.e., cell death). No relevant differences appeared except an increase in cell death with relaxin at two hours.

We claim:

1. A method of treating a cardiovascular indication comprising administering a natriuretic peptide, a diuretic peptide or a vasodilatory peptide to a patient in need thereof within 24 hours of clinical assessment of the patient.

2. The method of claim 1, comprising administering the natriuretic peptide to a patient in need thereof within 20 hours of clinical assessment of the patient.

3. The method of claim 1, comprising administering the natriuretic peptide to a patient in need thereof within 16 hours of clinical assessment of the patient.

4. The method of claim 1, comprising administering the natriuretic peptide to a patient in need thereof within 12 hours of clinical assessment of the patient.

5. The method of claim 1, comprising administering the natriuretic peptide to a patient in need thereof within 8 hours of clinical assessment of the patient.

6. The method of claim 1, wherein the natriuretic peptide is selected from the group consisting of atrial natriuretic peptide (ANP), brain natriuretic peptide (BNP), neseritide, C-type natriuretic peptide (CNP), dendroaspis natriuretic peptide (DNP), and urodilatin.

7. The method of claim 6, wherein the natriuretic peptide is neseritide.

8. The method of claim 6, wherein the natriuretic peptide is ularitide.

9. The method of claim 1, wherein the cardiovascular indication is heart failure, acute heart failure, chronic heart failure, congestive heart failure, acute decompensated heart failure, abnormal fluid accumulation in the heart, myocardial edema and dypsnea.

10. The method of claim 9, wherein the cardiovascular indication is acute decompensated heart failure.

11. The method of claim 1, wherein the natriuretic peptide is administered intravenously.

12. The method of claim 11, wherein the natriuretic peptide is administered for a time period between about 12 hours and 120 hours.

13. The method of claim 11, wherein the time period is between about 24 hours and about 96 hours.

14. The method of claim 11, wherein the time period is between about 24 and 72 hours.

15. The method of claim 11, wherein the time period is between about 36 and 60 hours.

16. The method of claim 11, wherein the time period is between about 40 and 56 hours.

17. The method of claim 11, wherein the time period is between about 44 and 52 hours.

18. The method of claim 11, wherein the time period is between about 46 and 50 hours.

19. The method of claim 11, wherein the time period is about 48 hours.

20. The method of claim 8, wherein the urodilatin is administered at a rate of at least 7.5 ng/kg/minute.

21. The method of claim 8, wherein the urodilatin is administered at a rate of 7.5 ng/kg/minute.

22. The method of claim 12, wherein the urodilatin is administered at a rate of 15 ng/kg/minute.

23. The method of claim 12, wherein the urodilatin is administered at a rate of 30 ng/kg/minute.

24. The method of claim 12, wherein the urodilatin is administered at a rate of 45 ng/kg/minute.

25. The method of claim 12, wherein the urodilatin is administered at a rate of 60 ng/kg/minute.

26. The method of claim 12, wherein the urodilatin is administered at a rate of 100 ng/kg/minute.

27. The method of claim 12, wherein the urodilatin is administered at a rate of 200 ng/kg/minute.

28. The method of claim 1, wherein the urodilatin is administered at the rate of 15 ng/kg/minute for a time period of about 48 hours.

29. The method of claim 1, wherein the vasodilatory peptide is relaxin.

30. The method of claim 1, wherein the administration prevents or minimizes myocardial cell death.

31. The method of claim 30, wherein the administration prevents or minimizes myocardial cell death in the presence of one or more factors selected from C-reactive protein, TNF-alpha, IL-1β, endothelin-1 or galectin-3.

32. The method of claim 1, wherein the administration prevents or minimizes the nitrosylation of myocardial cells.

33. The method of claim 32, wherein the administration prevents or minimizes the nitrosylation of myocardial cells in the presence of one or more factors selected from C-reactive protein, TNF-alpha, IL-1β, endothelin-1 or galectin-3.

34. A method of treating a cardiovascular indication comprising administering ularitide to a patient in need thereof within 24 hours of clinical assessment of the patient.

35. A method of treating a cardiovascular indication comprising administering a relaxin to a patient in need thereof within 24 hours of clinical assessment of the patient.
